(19) United States
(12) Patent Application Publication
(10) Pub. No.: US 2024/0393331 A1
(43) Pub. Date: Nov. 28, 2024

(54) MULTIPLEXED ELECTRONIC IMMUNOASSAY USING ENZYMATICALL AMPLIFIED METALLIZATION ON NANOSTRUCTURED SURFACES

(71) Applicant: Georgia Tech Research Corporation, Atlanta, GA (US)

(72) Inventors: Neda Rafat, Atlanta, GA (US); Aniruddh Sarkar, Atlanta, GA (US); Hanhao Zhang, Atlanta, GA (US)

(21) Appl. No.: 18/694,503

(22) PCT Filed: Sep. 23, 2022
(86) PCT No.: PCT/US2022/076924

§ 371 Date: Mar. 22, 2024

Related U.S. Application Data
(60) Provisional application No. 63/247,515, filed on Sep. 23, 2021.

(51) Int. Cl. G01N 33/569; G01N 33/543

(52) U.S. Cl. CPC G01N 33/56983; G01N 33/54346; G01N 33/5438; G01N 2333/165; G01N 2469/20

(57) ABSTRACT

An exemplary embodiment of the present disclosure provides a system for detecting biomarkers. The system can comprise a composition a first electrode, a second electrode, and an electrical property sensor in electrical communication with the first electrode and the second electrode. The electrical property sensor can be configured to measure an electrical property of the composition. The composition can comprise a molecule of interest configured to bind with a target molecule in a biological sample, a probe molecule configured to bind with the target molecule, and a first metal particle configured to bind with the probe molecule.

### CROSS-REFERENCE TO RELATED APPLICATIONS

This application claims the benefit of U.S. Provisional Application Ser. No. 63/247,515, filed on 23 Sep. 2021, which is incorporated herein by reference in its entirety as if fully set forth below.

### FEDERALLY SPONSORED RESEARCH STATEMENT

This invention was made with government support under grant/award number NIH 1R01AI152158-01 awarded by the National Institutes of Health and grant/award number NIH R01AI151178 awarded by the National Institutes of Health. The government has certain rights in the invention.

### FIELD OF THE DISCLOSURE

The various embodiments of the present disclosure relate generally to medical diagnostics, and more particularly to immunoassay based diagnostic tests.

### BACKGROUND

Aspects of the disclosed technology improve immunoassays. Immunoassays, such as enzyme-linked immunosorbent assays (ELISA) are widely used in biological research and clinical diagnostics as they provide excellent sensitivity, speed, and extended dynamic range due to their enhanced binding kinetics and ease of automation. They also enable highly multiplexed assays via the simultaneous measurement of multiple markers from a single sample using barcoded beads. However, many current assays require expensive and bulky optical instrumentation (e.g., lasers/photomultiplier tubes, etc.).

These immunoassays can require the use of expensive fluorescent markers and optical elements for readout. Optical elements are not as tough or durable as electrical elements and can be more difficult to transport. The optical detection systems required, such as flow cytometers are complex, bulky and expensive as they rely on lasers, photodetectors, and beam shaping lenses. This hinders their use especially in the context of widespread infectious diseases such as COVID-19.

Cheap, durable, and widespread diagnostic methods are needed for to track the spread of infectious diseases. Inexpensive yet sensitive and specific biomarker detection is a critical bottleneck in diagnostics, monitoring, and surveillance of infectious diseases such as COVID-19. Multiplexed detection of several biomarkers can achieve wider diagnostic applicability, accuracy, and ease-of-use, while reducing cost.

### BRIEF SUMMARY

An exemplary embodiment of the present disclosure provides a composition comprising a molecule of interest configured to bind with a target molecule in a biological sample, a probe molecule configured to bind with the target molecule, a first metal particle configured to bind with the probe molecule, and a second metal particle configured to amplify a binding of the first metal particle.

In any of the embodiments disclosed herein, the first metal particle can comprise one or more of: a silver nanoparticle, a gold nanoparticle, an iron nanoparticle, silver, gold, iron, and platinum.

In any of the embodiments disclosed herein, the second metal particle can comprise one or more of: gold, platinum, and iron oxide.

In any of the embodiments disclosed herein, the molecule of interest can comprise one or more of: an antigen, an epitope, an antibody, protein, DNA, RNA, virus, bacterium or mammalian cell.

In any of the embodiments disclosed herein, the target molecule can comprise one or more of: an antibody, a paratope, a protein, DNA, RNA, a virus, a bacterium, or mammalian cell.

In any of the embodiments disclosed herein, the probe molecule can further comprise one or more of: horseradish peroxidase, alkaline phosphatase.

In any of the embodiments disclosed herein, the biological sample can comprise one or more of: serum, whole blood, saliva, urine, cerebrospinal fluid.

In any of the embodiments disclosed herein, the composition can further comprise a buffer. In any of the embodiments disclosed herein, the buffer can be a phosphate buffer having a pH of from about 6.0 to about 7.0. In any of the embodiments disclosed herein, the buffer can be a phosphate buffer comprising a pH of approximately 6.4.

An exemplary embodiment of the present disclosure provides a method for measuring biomarkers, the method comprising forming an electrical connection between a first electrode and a second electrode and measuring an electrical property of the electrical connection. The electrical property can be indicative of the presence or absence of a biomarker.

In any of the embodiments disclosed herein, forming the electrical connection can comprise allowing a molecule of interest disposed between the first electrode and the second electrode to bind with a target molecule in a biological sample, allowing a probe molecule to bind with the target molecule that is bound to the molecule of interest, and allowing a first metal particle to bind with the probe molecule that is bound to the target molecule to modify the electrical property of the electrical connection.

In any of the embodiments disclosed herein, allowing the molecule of interest to bind with the target molecule can comprise incubating the molecule of interest in a sample solution comprising the biological sample.

In any of the embodiments disclosed herein, incubating the molecule of interest in the sample solution lasts for approximately 1800 seconds at approximately room temperature.

In any of the embodiments disclosed herein, allowing the probe molecule to bind with the target molecule can comprise incubating the target molecule in a probe solution comprising the probe molecule.

In any of the embodiments disclosed herein, incubating the target molecule in the probe solution lasts for approximately 3600 seconds at approximately room temperature.

In any of the embodiments disclosed herein, the probe solution can further comprise a second metal particle. In any of the embodiments disclosed herein, the second metal particle can be configured to amplify the forming the electrical connection. In any of the embodiments disclosed herein, the second metal particle can comprise one or more of: gold, platinum, and iron oxide.

In any of the embodiments disclosed herein, the first electrode and second electrode can be disposed on a plate. In any of the embodiments disclosed herein, the plate can have on its surface a second metal particle configured to amplify the forming the electrical connection.

In any of the embodiments disclosed herein, allowing the first metal particle to bind with the probe molecule that is bound to the target molecule can comprise enzymatically depositing the first metal particle on the probe molecule.

In any of the embodiments disclosed herein, the first metal particle can comprise one or more of: a silver nanoparticle, a gold nanoparticle, an iron nanoparticle, silver, gold, iron, and platinum.

In any of the embodiments disclosed herein, measuring the electrical property of the electrical connection can comprise measuring an impedance across the electrical connection.

In any of the embodiments disclosed herein, measuring the impedance across the electrical connection can be performed with a lock-in amplifier.

In any of the embodiments disclosed herein, measuring the impedance can comprise sampling at approximately 100,000 samples per second at one or more frequencies. In any of the embodiments disclosed herein, the one or more frequencies can be between approximately 45 kilohertz and 35 megahertz.

An exemplary embodiment of the present disclosure provides a system for detecting biomarkers. The system can comprise a composition a first electrode, a second electrode, and an electrical property sensor in electrical communication with the first electrode and the second electrode. The electrical property sensor can be configured to measure an electrical property of the composition. The composition can comprise a molecule of interest configured to bind with a target molecule in a biological sample, a probe molecule configured to bind with the target molecule, and a first metal particle configured to bind with the probe molecule.

In any of the embodiments disclosed herein, the composition can comprise a second metal particle configured to amplify a binding of the first metal particle.

In any of the embodiments disclosed herein, the system can further comprise a plate, and the first electrode and the second electrode can be disposed on the plate within a well configured to contain liquid.

In any of the embodiments disclosed herein, the well can be formed in a film.

In any of the embodiments disclosed herein, the plate can be polylysine coated glass.

In any of the embodiments disclosed herein, the plate can have on its surface the second metal particle.

In any of the embodiments disclosed herein, the first metal particle can be configured to form an electrical connection between the first electrode and the second electrode.

In any of the embodiments disclosed herein, the electrical property can comprise an impedance across the electrical connection.

In any of the embodiments disclosed herein, the system can further comprise a processor configured to determine a concentration of the target molecule in the biological sample based on the impedance across the electrical connection.

In any of the embodiments disclosed herein, the electrical property sensor comprises a lock-in amplifier.

In any of the embodiments disclosed herein, the biological sample can comprise one or more of: serum, whole blood, saliva, urine, cerebrospinal fluid.

In any of the embodiments disclosed herein, the system can further comprise a buffer.

In any of the embodiments disclosed herein, the system can further comprise a sample solution comprising the biological sample.

In any of the embodiments disclosed herein, the system can further comprise a probe solution comprising the probe molecule. In any of the embodiments disclosed herein, the probe solution further comprising a second metal particle configured to amplify a binding of the first metal particle.

In any of the embodiments disclosed herein, the first metal particle can comprise one or more of: a silver nanoparticle, a gold nanoparticle, an iron nanoparticle, silver, gold, iron, and platinum.

In any of the embodiments disclosed herein, the first electrode can be an interdigitated electrode comprising a first plurality of fingers, and the second electrode can be an interdigitated electrode comprising a second plurality of fingers.

In any of the embodiments disclosed herein, at least one of the first plurality of fingers can be adjacent to at least one of the second plurality of fingers and separated by a gap, and the gap can be approximately 5 micrometers.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 provides an illustrated view of a composition, in accordance with an exemplary embodiment of the present disclosure.

FIG. 2 provides a magnified view of a metal particle, in accordance with an exemplary embodiment of the present disclosure.

FIG. 3 provides an illustrated view of an electrical connection and the forming thereof, in accordance with exemplary embodiments of the present disclosure.

FIG. 4 provides an illustrated view of a composition, in accordance with exemplary embodiments of the present disclosure.

FIG. 5 provides a flow chart of a method for measuring biomarkers, in accordance with an exemplary embodiment of the present disclosure.

FIG. 6 provides a flow chart of a method for measuring biomarkers, in accordance with an exemplary embodiment of the present disclosure.

FIG. 7 provides an exploded view of a plate, a film, and a magnified view of a first electrode and a second electrode, in accordance with an exemplary embodiment of the present disclosure.

FIG. 8 provides a perspective view of a well, a film, a plate, and electrodes in a system for detecting biomarkers, in accordance with an exemplary embodiment of the present disclosure.

FIG. 9 provides an exploded view of a well, a film, a plate, and electrodes in a system for detecting biomarkers, in accordance with an exemplary embodiment of the present disclosure.

FIG. 10 provides top view of interdigitated electrodes in a system for detecting biomarkers, in accordance with an exemplary embodiment of the present disclosure.

FIG. 11A provides a perspective view of a system for detecting biomarkers, in accordance with an exemplary embodiment of the present disclosure.

FIG. 11B provides a perspective view of a system for detecting biomarkers, in accordance with an exemplary embodiment of the present disclosure.

FIG. 12 provides a top view of a interdigitated electrodes and an electrical connection, in accordance with an exemplary embodiment of the present disclosure.

FIG. 13 provides plots of impedance and absorbance measured with a system for detecting biomarkers, in accordance with an exemplary embodiment of the present disclosure.

FIG. 14 provides plots of impedance measured with a system for detecting biomarkers, in accordance with an exemplary embodiment of the present disclosure.

FIG. 15 provides a plot of impedance versus dilution factor, in accordance with an exemplary embodiment of the present disclosure.

FIG. 16 provides plots of impedance for various biomarkers as measured with a system for detecting biomarkers, in accordance with an exemplary embodiment of the present disclosure.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

It must be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms a, an, and the do not denote a limitation of quantity, but rather denote the presence of at least one of the referenced item.

As used herein, the term “and/or” may mean “and,” it may mean “or,” it may mean exclusive-or” it may mean “one,” it may mean “some, but not all,” it may mean “neither,” and/or it may mean “both.” The term “or” is intended to mean an inclusive “or.”

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose. It is to be understood that embodiments of the disclosed technology may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Similarly, as used herein, “substantially free” of something, or “substantially pure”, and like characterizations, can include both being “at least substantially free” of something, or “at least substantially pure”, and being “completely free” of something, or “completely pure.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

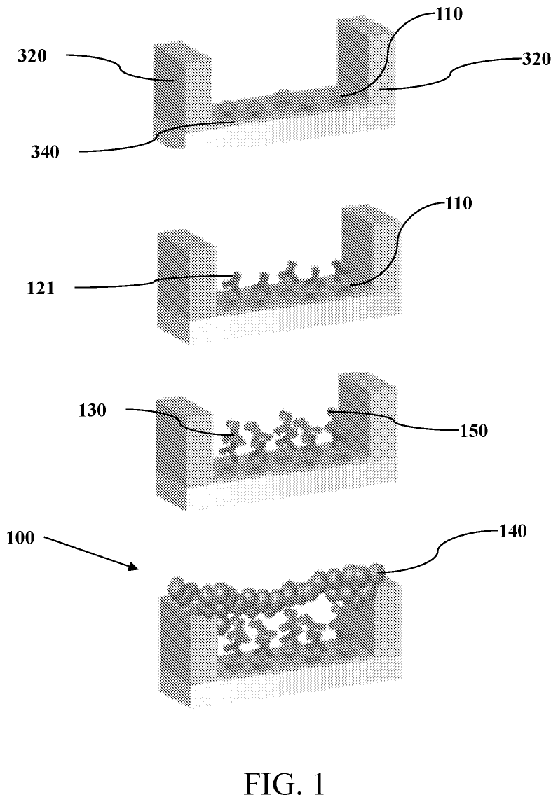

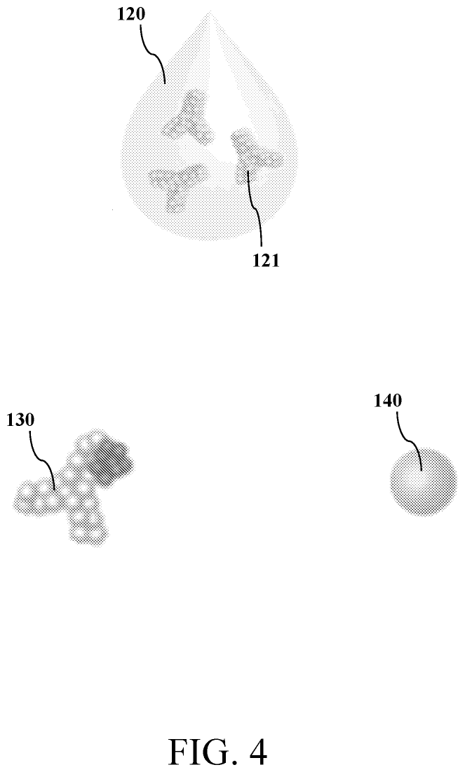

As shown in FIGS. 1-4, an exemplary embodiment of the present disclosure provides a composition (100) comprising a molecule of interest (110) configured to bind with a target molecule (121) in a biological sample (120), a probe molecule (130) configured to bind with the target molecule (121), a first metal particle (140) configured to bind with the probe molecule (130), and a second metal particle (150) configured to amplify a binding of the first metal particle (140).

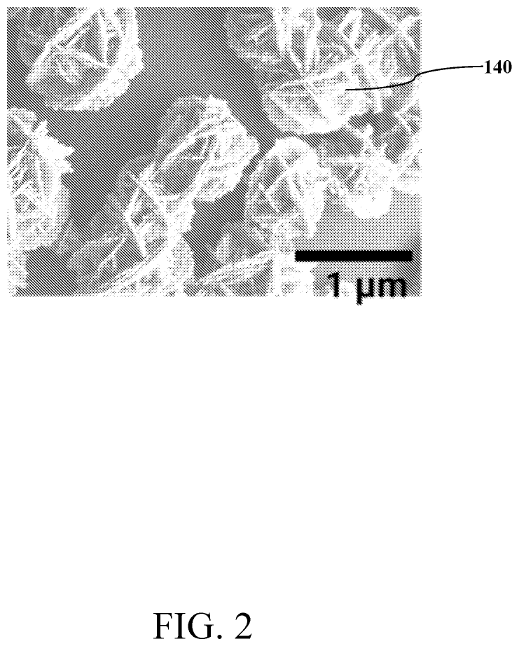

As shown in FIG. 2, in any of the embodiments disclosed herein, the first metal particle (140) can comprise one or more of: a silver nanoparticle, a gold nanoparticle, an iron nanoparticle, silver, gold, iron, and platinum.

In any of the embodiments disclosed herein, the second metal particle (150) can comprise one or more of: gold, iron oxide, and platinum.

In any of the embodiments disclosed herein, the molecule of interest (110) can comprise one or more of: an antigen, an epitope, an antibody, protein, DNA, RNA, virus, bacterium, or mammalian cell.

In any of the embodiments disclosed herein, the target molecule (121) can comprise one or more of: an antibody, a paratope, a protein, DNA, RNA, a virus, a bacterium, mammalian cell.

In any of the embodiments disclosed herein, the probe molecule (130) can further comprise horseradish peroxidase.

In any of the embodiments disclosed herein, the biological sample (120) can comprise one or more of: serum, whole blood, saliva, urine, cerebrospinal fluid.

In any of the embodiments disclosed herein, the composition can further comprise a buffer. In any of the embodiments disclosed herein, the buffer can be a phosphate buffer having a pH of from about 6.0 to about 7.0. In any of the embodiments disclosed herein, the buffer can be a phosphate buffer comprising a pH of approximately 6.4.

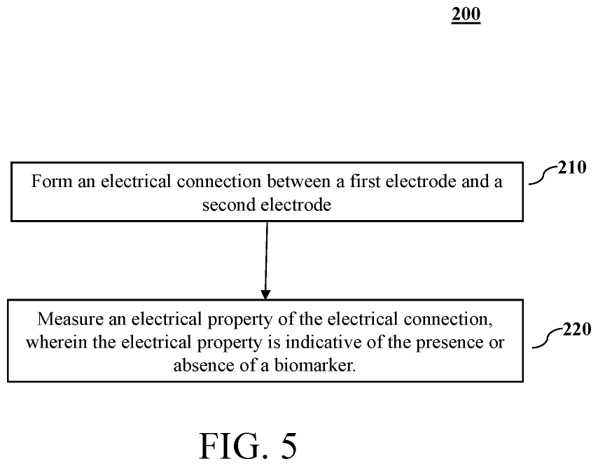

As shown in FIG. 5, an exemplary embodiment of the present disclosure provides a method (200) for measuring biomarkers, the method comprising forming (210) an electrical connection (310) between a first electrode (320) and a second electrode (330) and measuring (220) an electrical property (311) of the electrical connection (310). The electrical property (311) can be indicative of the presence or absence of a biomarker.

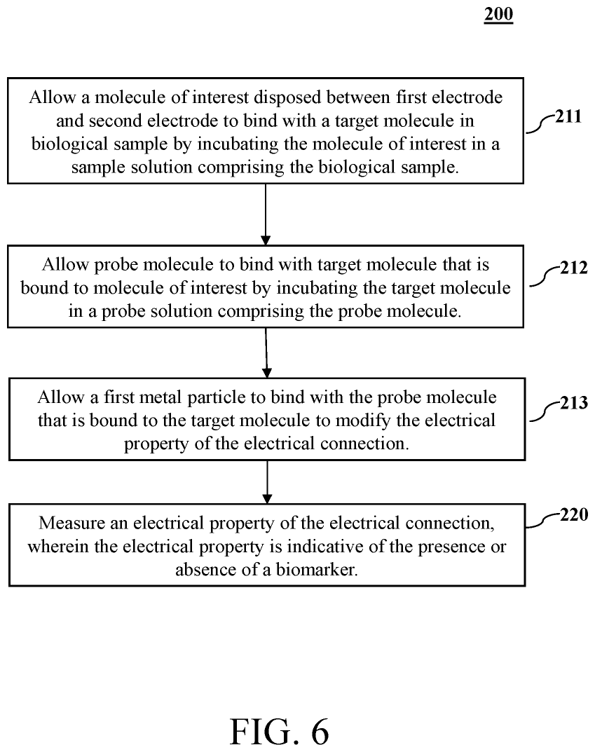

As shown in FIG. 6, in any of the embodiments disclosed herein, forming the electrical connection (310) can comprise allowing (211) a molecule of interest (110) disposed between the first electrode (320) and the second electrode (330) to bind with a target molecule (121) in a biological sample (120), allowing (212) a probe molecule (130) to bind with the target molecule (121) that is bound to the molecule of interest (110), and allowing (213) a first metal particle (140) to bind with the probe molecule (130) that is bound to the target molecule (121) to modify the electrical property (311) of the electrical connection (310).

In any of the embodiments disclosed herein, allowing the molecule of interest (110) to bind with the target molecule (121) can comprise incubating the molecule of interest (110) in a sample solution comprising the biological sample (120).

In any of the embodiments disclosed herein, incubating the molecule of interest (110) in the sample solution lasts for approximately 1800 seconds at approximately room temperature.

In any of the embodiments disclosed herein, allowing the probe molecule (130) to bind with the target molecule (121) can comprise incubating the target molecule (121) in a probe solution comprising the probe molecule (130).

In any of the embodiments disclosed herein, incubating the target molecule (121) in the probe solution lasts for approximately 3600 seconds at approximately room temperature.

In any of the embodiments disclosed herein, the probe solution can further comprise a second metal particle (150). In any of the embodiments disclosed herein, the second metal particle (150) can be configured to amplify the forming the electrical connection (310). In any of the embodiments disclosed herein, the second metal particle (150) can be gold.

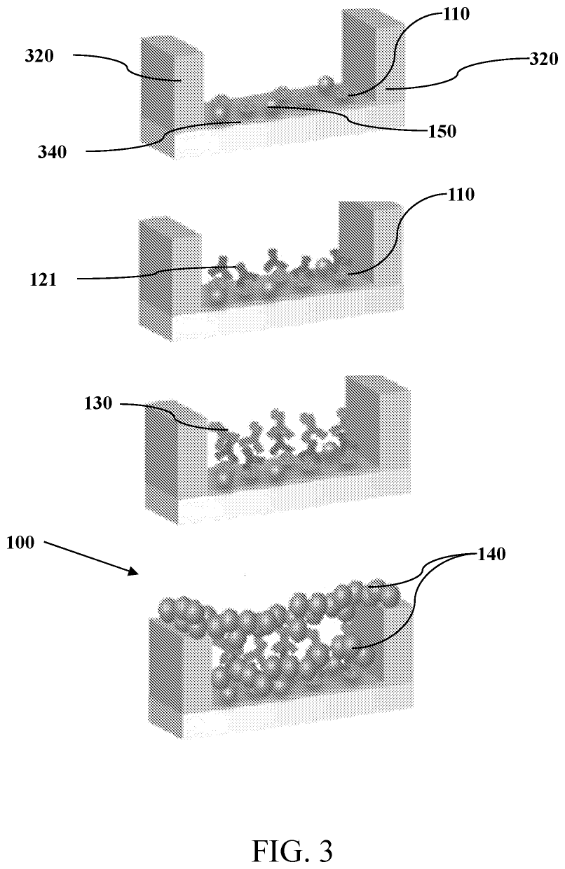

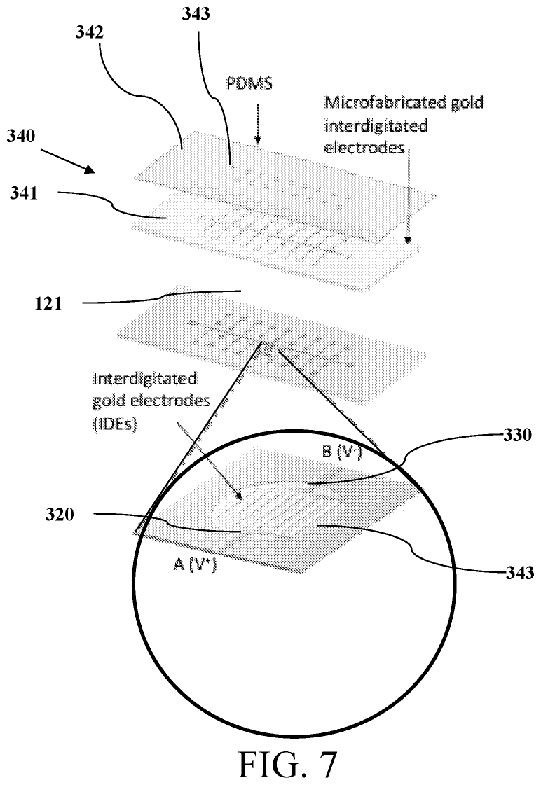

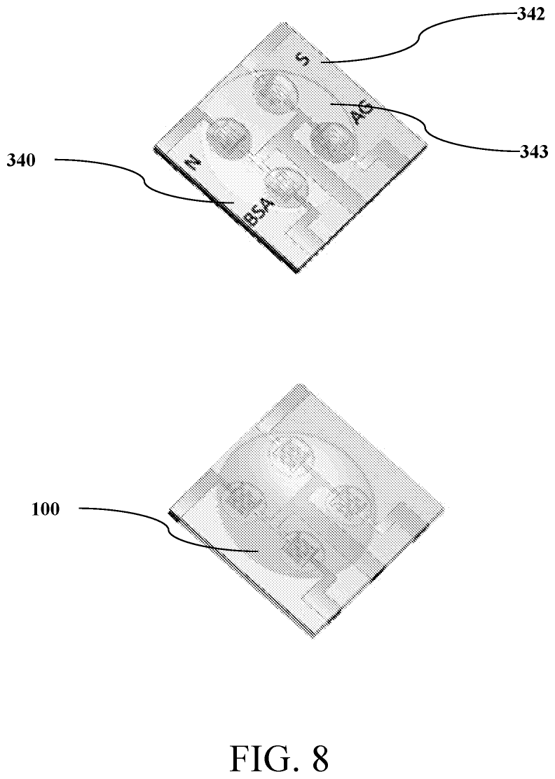

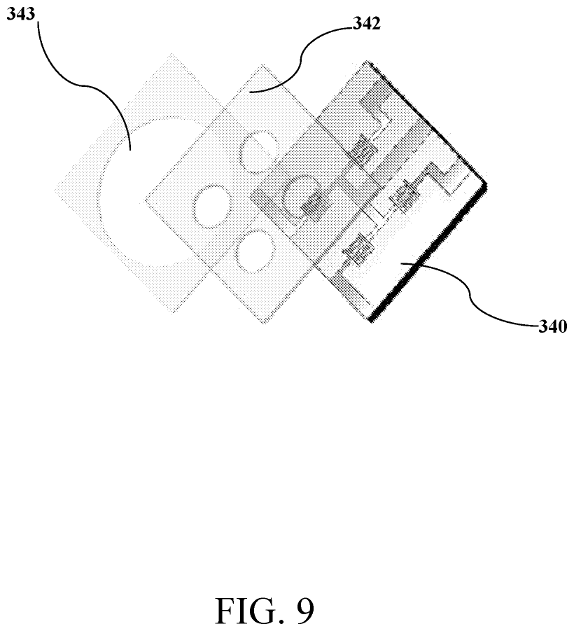

As shown in FIGS. 7-9. in any of the embodiments disclosed herein, the first electrode (320) and second electrode (330) can be disposed on a plate. As shown in FIG. 3, in any of the embodiments disclosed herein, the plate (340) can have on its surface (341) a second metal particle (150) configured to amplify the forming the electrical connection (310).

In any of the embodiments disclosed herein, allowing (211) the first metal particle (140) to bind with the probe molecule (130) that is bound to the target molecule (121) can comprise enzymatically depositing the first metal particle (140) on the probe molecule (130).

In any of the embodiments disclosed herein, the first metal particle (140) can comprise one or more of: a silver nanoparticle, a gold nanoparticle, an iron nanoparticle, silver, gold, iron, and platinum.

In any of the embodiments disclosed herein, measuring (220) the electrical property (311) of the electrical connection (310) can comprise measuring an impedance across the electrical connection (310).

In any of the embodiments disclosed herein, measuring the impedance across the electrical connection (310) can be performed with a lock-in amplifier.

In any of the embodiments disclosed herein, measuring (220) the impedance can comprise sampling at approximately 100,000 samples per second at one or more frequencies. In any of the embodiments disclosed herein, the one or more frequencies can be between approximately 45 kilohertz and 35 megahertz.

An exemplary embodiment of the present disclosure provides a system (300) for detecting biomarkers. As shown in FIG. 1, FIG. 3, and FIGS. 7-10, the system (300) can comprise a composition a first electrode (320), a second electrode (330), and an electrical property sensor (350) in electrical communication with the first electrode (320) and the second electrode (330). The electrical property sensor (350) can be configured to measure an electrical property of the composition (100). The composition can comprise a molecule of interest (110) configured to bind with a target molecule (121) in a biological sample (120), a probe molecule (130) configured to bind with the target molecule (121), and a first metal particle (140) configured to bind with the probe molecule (130).

In any of the embodiments disclosed herein, the composition (100) can comprise a second metal particle (150) configured to amplify a binding of the first metal particle (140).

As shown in FIG. 7-9, in any of the embodiments disclosed herein, the system can further comprise a plate (340), and the first electrode (320) and the second electrode (330) can be disposed on the plate (340) within a well (343) configured to contain liquid. In any of the embodiments disclosed herein, the well (343) can be formed in a film (342). In any of the embodiments disclosed herein, the plate (340) can be polylysine coated glass.

As shown in FIG. 2, in any of the embodiments disclosed herein, the plate (340) can have on its surface (341) the second metal particle (150).

In any of the embodiments disclosed herein, the first metal particle (140) can be configured to form an electrical connection (310) between the first electrode (320) and the second electrode (330).

In any of the embodiments disclosed herein, the electrical property can comprise an impedance across the electrical connection (310).

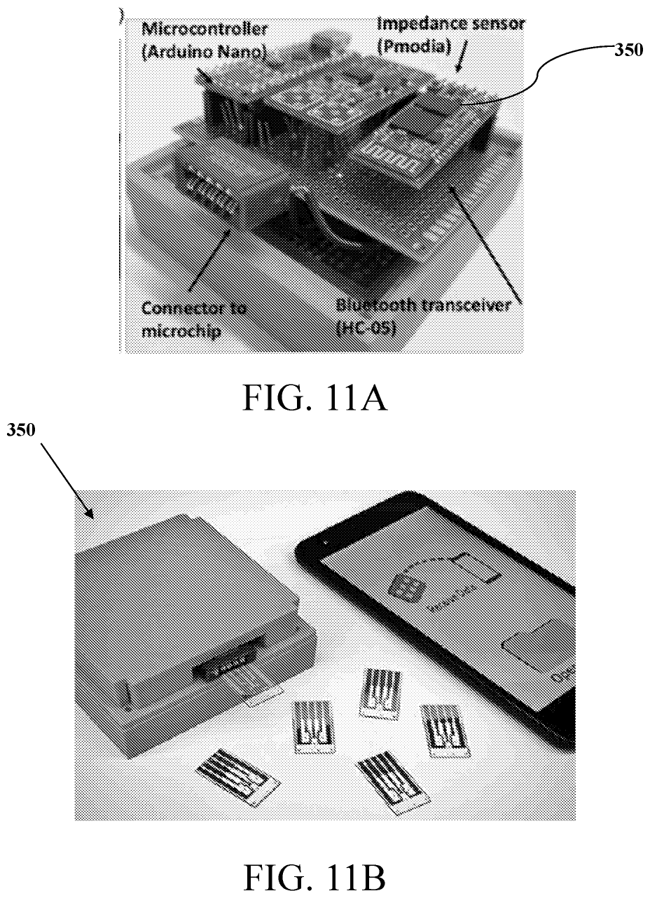

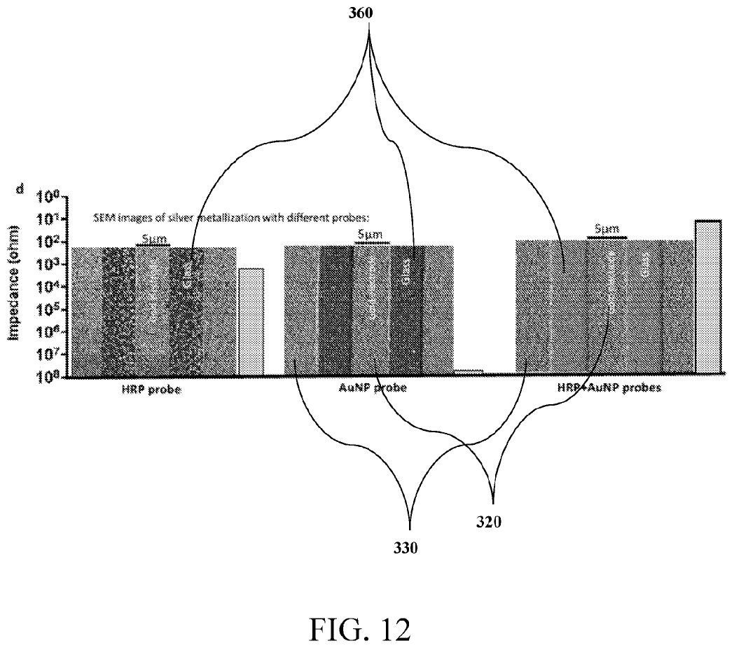

As shown in FIG. 11A, in any of the embodiments disclosed herein, the system can further comprise a processor configured to determine a concentration of the target molecule (121) in the biological sample (120) based on the impedance across the electrical connection (310).

In any of the embodiments disclosed herein, the electrical property sensor (350) comprises a lock-in amplifier.

In any of the embodiments disclosed herein, the biological sample (120) can comprise one or more of: serum, whole blood, saliva, urine, cerebrospinal fluid.

In any of the embodiments disclosed herein, the system can further comprise a buffer.

In any of the embodiments disclosed herein, the system can further comprise a sample solution comprising the biological sample (120).

In any of the embodiments disclosed herein, the system can further comprise a probe solution comprising the probe molecule (130). In any of the embodiments disclosed herein, the probe solution further comprising a second metal particle (150) configured to amplify a binding of the first metal particle (140).

In any of the embodiments disclosed herein, the first metal particle (140) can comprise one or more of: a silver nanoparticle, a gold nanoparticle, an iron nanoparticle, silver, gold, iron, and platinum.

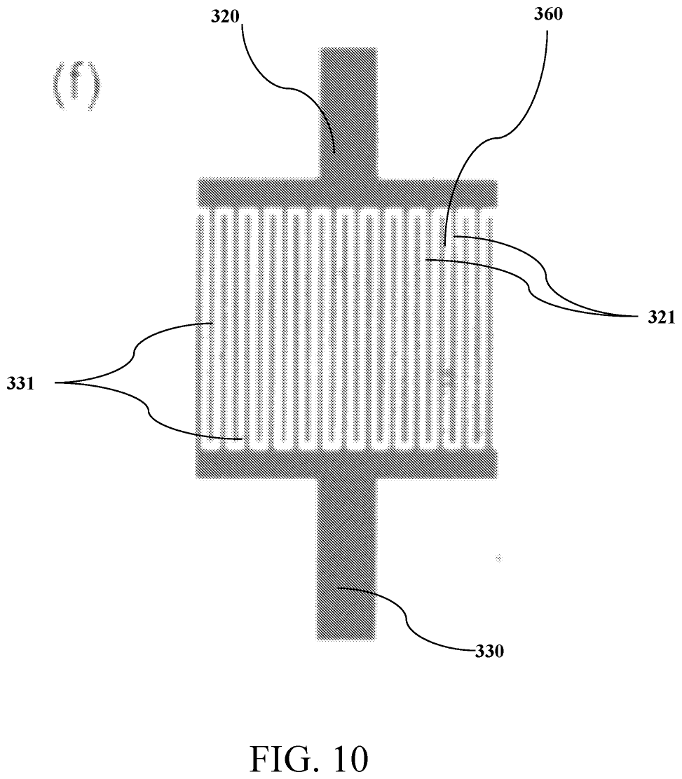

As shown in FIG. 10, in any of the embodiments disclosed herein, the first electrode (320) can be an interdigitated electrode comprising a first plurality of fingers (321), and the second electrode (330) can be an interdigitated electrode comprising a second plurality of fingers (331).

In any of the embodiments disclosed herein, at least one of the first plurality of fingers (321) can be adjacent to at least one of the second plurality of fingers (321) and separated by a gap (360), and the gap (360) can be approximately 5 micrometers.

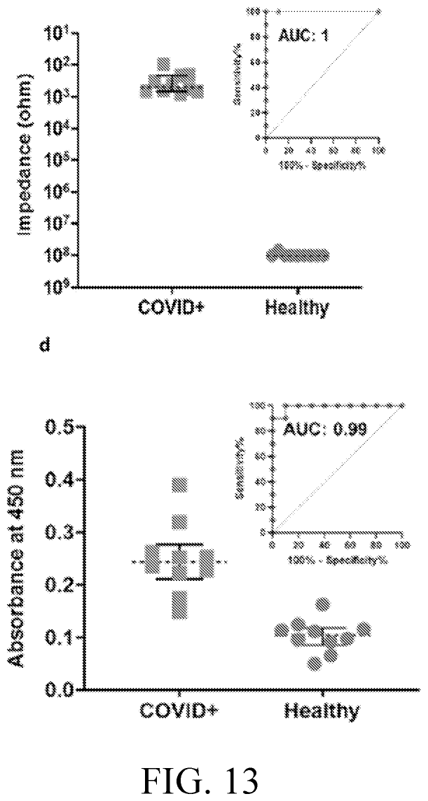

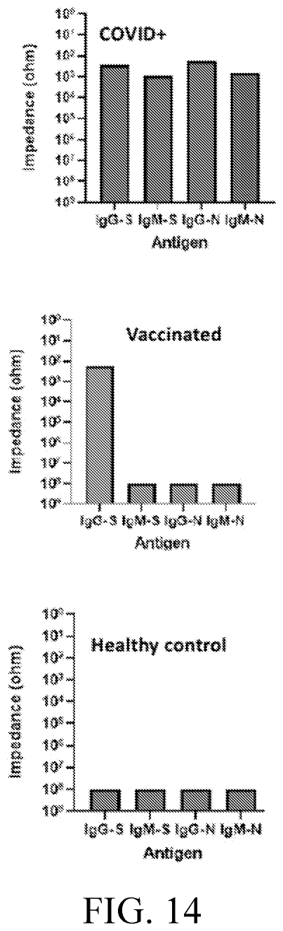

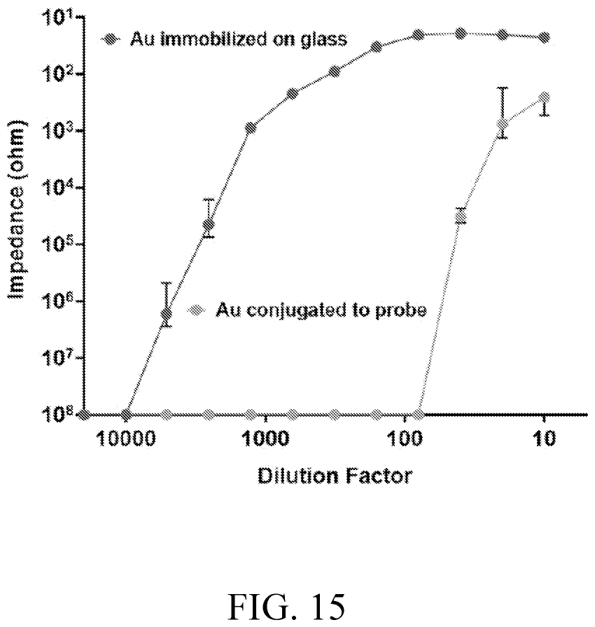

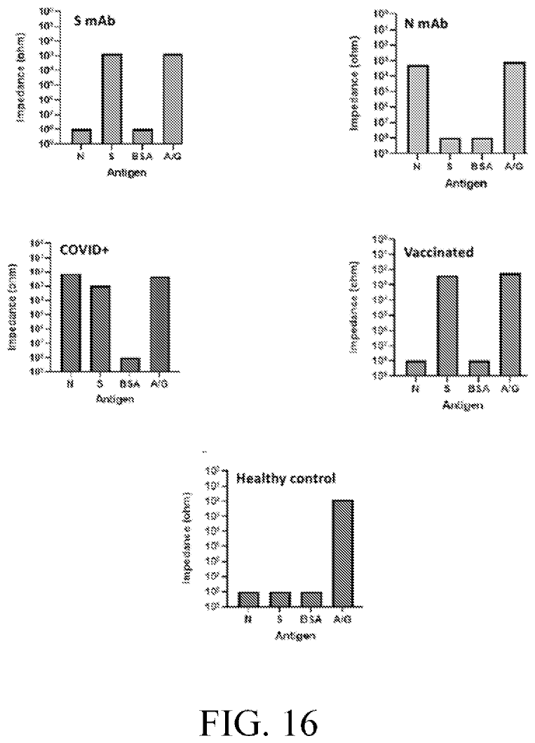

As summarized by the results in FIG. 13-16, the measurement of the electrical property can be used to determine the presence or absence of a biomarker. In any of the embodiments disclosed herein, lower impedance between the first electrode and the second electrode can indicate a higher degree of metallization and thus higher concentration of the biomarker.

The following examples further illustrate aspects of the present disclosure. However, they are in no way a limitation of the teachings or disclosure of the present disclosure as set forth herein.

### Examples

Below, exemplary compositions, methods, and systems for detecting and measuring biomarkers are disclosed. Disclosed herein is a signal transduction scheme that can convert the specific recognition or binding of a wide range of biomarkers to a quantitative electronic or optical signal by deposition of a metal layer in a biomarker concentration-dependent manner. This technique exploits a synergistic effect of probe-directed enzymatic activity and catalytic activity of nanomaterials such as nanostructured surfaces to deposit an amplified metal layer whose electrical property or optical density can be easily and inexpensively measured for quantification of biomarkers. Furthermore, the localized nature of this metal deposition enables multiplexed biomarker measurements from a small sample volume.

Enzyme-linked immunosorbent assays (ELISAs) are commonly used for biomarker detection. However, they require expensive and bulky instrumentation and are restricted to laboratory environments, and not suitable for point-of-care applications. Besides, they can only detect single biomarkers at a time while multiplexed detection of biomarkers in a disease can provide a more comprehensive diagnosis.

Here, for rapid electronic biomarker detection, nanomaterials and biomarker-specific capture agents are immobilized on a solid surface between two microelectrodes. After the addition of the biomarker-containing sample, an enzyme-labeled probe is added. Finally, a mixture of metal-containing enzymatic substrates is added to deposit localized metal on a substrate that connects the microelectrodes enabling electronic measurement of the biomarker concentration. Additionally, the deposited metal layer also blocks and/or reflects light and is visible to the naked eye as well as amenable to simple optical measurement, for example, using a cellphone camera.

The current COVID-19 pandemic and other recent outbreaks such as Ebola, MERS, SARS and H1N1 have underscored the need for early detection and continued surveillance of emerging and re-emerging infectious diseases. Outbreaks, many of which start with a zoonotic transmission event, often begin in remote or resource-poor areas but threaten to rapidly spread globally if not brought under control promptly. A critical bottleneck in achieving this is the lack of rapid and scalable yet accurate diagnostic, monitoring and surveillance tools for infectious diseases. Diagnostic availability severely limited early efforts to contain the COVID-19 pandemic in many countries globally. Even now, as the worldwide deployment of COVID-19 vaccines progresses, concerns regarding the durability of vaccine efficacy, especially against newly emerging variants, have created a further need for the rapid monitoring of heterogenous and time-varying individual vaccine responses as well. Multiplexed detection of various biomarkers such as multiple infection or vaccine-elicited antigen-specific antibodies in a single test, especially when coupled to machine-learning based multivariate analytics to derive further clinical insight, can help fulfill the above critical needs. Enzyme-linked immunosorbent assays (ELISA) are the gold-standard in laboratory-based sensitive and quantitative detection of a range of biomarkers including serological testing for infectious diseases such as COVID-19. For example, titers of neutralizing antibodies directed against the Spike antigen of SARS-CoV-2, as measured by quantitative ELISAs, are key correlates of vaccine-induced protection against COVID-19 and can thus be used to monitor individual vaccine efficacy.

ELISAs are routinely performed using bulky and expensive but highly sensitive instruments which require highly trained personnel to operate. These instruments are usually based on optical detection of enzymatic probe-catalyzed amplification products using, for example, lasers for illumination and photomultiplier tubes for detection. They remain too complex and expensive to scale and deploy globally. Even in resource-rich settings they are currently used only in centralized diagnostics laboratories. On the other end of the complexity versus cost space, inexpensive lateral flow-based assays (LFA) are easier to perform and deploy as point-of-care (POC) tests which offer binary (yes/no) readouts. These tests however lack both the sensitivity and quantitative ability of ELISAs. Additionally, neither ELISAs nor LFAs routinely offer multiplexing ability and require running separate tests for each separate biomarker further increasing cost and complexity. In blood-based tests, the need for a larger sample volume for multiple tests can pose a significant practical challenge to POC testing. Large volumes of blood (>1 mL) can only be obtained via venipuncture-based phlebotomy, instead of the finger-prick based acquisition of small volume, droplet-scale (<10 μL) samples. This necessitates additional equipment, expertise (e.g., trained phlebotomists) and results in higher biosafety and regulatory burdens as well.

Electrical and electrochemical detection methods can transduce biochemical information such as analyte concentration directly to an electronic signal and thus offer an attractive opportunity to bypass expensive optical detection methods while retaining sensitivity and quantitative ability. Electronic detection principles are also more amenable to scaling down sample, sensor and system size and cost via miniaturization, using microfabrication techniques, without the loss of sensitivity that some optical detection methods (e.g., absorbance) suffer upon length scaling. Most current electrochemical biosensors however still remain too complex, specialized and expensive compared to LFAs and are thus have not found wide use in the clinic for POC diagnostics. Silver reduction catalyzed by gold nanoparticles (AuNP) attached to target-specific probes has been utilized earlier for POC optical detection of infectious disease biomarkers as well for electrical conductivity-based DNA detection using passivated gold electrodes. Enzymatic silver metallization offers an attractive alternative for localized and rapid enzymatically enhanced silver deposition which can then be seamlessly coupled to a broad range of ELISA chemistries. However, the electrical properties of enzymatically deposited silver layers have remained under-explored and difficult to control.

An exemplary embodiment, according to the disclosure herein, is a broadly applicable, miniaturized electronic detection principle for bioassays which can retain the simplicity of signal readout such as LFAs and yet can offer the sensitivity and quantitative detection ability of ELISAs while adding the ability for multiplexed detection of biomarkers. Using surface-bound AuNPs to create a catalytic nanostructured surface and adding enzyme-labeled target-specific probes, there is a synergistic effect on the probe-directed enzymatic metallization which causes significantly enhanced selective silver deposition resulting in a highly conducting silver metal layer. The binding of biomarkers can be converted directly to electrical properties of the amplified silver layer which can then be measured simply as a dry-phase resistance using microelectrodes without the use of any bulky intermediate optics or expensive instruments. This method can be used for sensitive and quantitative detection of antibodies against SARS-CoV-6 2 viral antigens from convalescent COVID-19 patient serum.

The miniaturized nature of this detection technique can be used in microchips which enable high-throughput clinical screening bioassays in a portable format allowing the measurement of larger numbers (>50) of small volumes of (<0.1 μL) clinical samples on a single chip.

Finally, the localized nature of the metallization reaction and its dry-phase readout technique enables specific detection of multiple biomarkers on nearby but electrically unconnected microelectrode pairs, from a single sub-5 μL droplet of sample as well.

To explore the electrical properties of enzymatically amplified silver metallization on micro-interdigitated electrodes (μIDEs), initial assays of horseradish peroxidase-conjugated streptavidin (HRP-SA) binding with biotin-conjugated bovine serum albumin (biotin-BSA) immobilized on the μIDEs as a model target-probe binding pair can serve as an example. For this, a microchip with an array of gold μIDEs on glass is microfabricated, which have very high electrical resistance (>108Ω) or effectively open circuits. These are then treated with a molecular adhesion layer (poly-l-lysine, PLL) and reversibly assembled with a thin laser-cut polydimethylsiloxane (PDMS) film with an array of microwells. Biotin-BSA is immobilized, as target, in the microwells incubated with the HRP-SA probe solution alone. Silver enzymatic metallization substrate solution is added next and after a set metallization reaction time, the microchip is washed and dried. Enzymatic silver metallization is observed on the microchip, but it is found, in optical micrographs, to have a low density and measurements of μIDE resistance showed a closed circuit but with a high (˜5×10+Ω) and non-repeatable (>10%) measured resistance. Electron microscopy revealed a low number density of silver nanoparticles on the glass between electrodes and also a characteristic ‘desert rose’ or ‘rosette’ morphology of individual silver nanoparticles. The low density of silver nanoparticles on the glass is not enough to create a highly electrically conductive and repeatable path between two successive fingers of the μIDEs. The high density of silver nanoparticles on gold electrodes is due to the fact that the gold electrode surface itself can act as a competing nucleating and catalytic surface for silver reduction and deposition.

Streptavidin-labeled AuNPs (AuNP-SA) can be included in the assays. In a control assay, AuNP-SA is used as the only probe to investigate the amount of silver metallization that can be catalyzed by AuNPs alone. Results of this AuNP-only assay showed, however, an even lower density silver metallization on both glass surface and gold electrodes resulting in a very high measured resistance or effectively an open circuit (>108Ω). Enzymatic metallization can create higher silver metallization than AuNPs alone, but neither alone is sufficient to create high conductivity paths between the electrodes. In an assay where a mixture of AuNP-SA and HRP-SA is used as probe, silver metallization is highly enhanced on the glass surface, and this results in a repeatable and low measured resistance (<100Ω). This forms a very high density of nanoparticles with similar rosette-like morphology formed in a highly connected network of nanoparticles which are merging to also show an almost continuous layer-like morphology. This high-density, highly connected, and thus highly conductive silver metallization is greater than either of the above two metallization reactions catalyzed by HRP alone or AuNP alone. The enhanced density of metallization is greater than what would be expected from a simple additive effect of the two independent reactions. The results of this assay with combined AuNP and HRP probes thus showed that the surface-bound AuNPs and the enzyme have a hitherto-undescribed synergistic catalytic effect on the metallization which significantly enhances its density and conductivity.

Silver metallization density on the surface depends on two key steps: reduction of silver ions and the attachment of the reduced silver to the surface. Both AuNPs and HRP are known to independently act as catalytic agents for reduction of silver ions in the presence of appropriate other reducing agents and for HRP, oxidizing substrates as well. AuNPs act as a nucleating surface for the deposition of the reduced silver metal. HRP—and indeed many other proteins—act as nucleating surface for the deposition of reduced silver metal.

The synergistic effect of AuNPs and HRP emerges from the fact the HRP-catalyzed reduced silver can, in this case, co-deposit on nearby AuNPs, using them as the nucleating surface rather than the HRP alone. This can relieve the blocking and self-limiting effect of the deposited silver on the catalytic effect of the HRP and allows the reduction reaction to proceed for much longer before the enzyme is rendered ineffective. Additionally, AuNPs continue to play a catalytic role as well in reducing more silver ions which adds to the total silver metallization on the surface.

This novel synergistic effect can be utilized to transduce a biochemical binding event to an enzymatically amplified, dry-stable, silver metallization layer and thus a simply measurable large change in electrical resistance. For detection of anti-viral antigen-specific antibodies in COVID-19, human IgG antibody directed against the SARS-CoV-2 Spike protein (anti-S IgG) is selected as the initial target biomarker to pursue this. To obtain a direct electrical readout of anti-S IgG, Spike protein (S) is immobilized on the μIDEs. Serially diluted clinical serum samples from convalescent COVID-19 patients are added to the microwells (3 μL per well) and then probed with a mixture of HRP-labeled and AuNP-labeled anti-human IgG antibodies followed by the silver metallization substrate solutions. Dense silver metallization occurs on the μIDEs which are also found to have a serum-dilution dependent measured while corresponding pre-pandemic healthy control serum and buffer only control showed little or no silver deposition
and remained open circuits. This demonstrates the ability of this transduction scheme to electronically detect and quantify viral antigen-specific antibodies as COVID-19 biomarkers from patient serum.

While the mixed AuNP-labeled probe and HRP-labeled probe-based immunoassay design clearly demonstrates the same synergistic enhanced silver metallization effect as described herein, to avoid the mixed probes creating a competition between the two probes for binding to the captured anti-S IgG molecules, which at high dilutions can act as a limiting reagent, and to further boost the sensitivity of this transduction strategy, a second variation of the assay scheme where which still integrates AuNPs in the assay but in an earlier assay step can be employed. In this second assay scheme, AuNPs are co-immobilized on μIDEs with the antigen (i.e. S protein) in the first step. This results in the creation of a nanostructured, catalytic AuNP-bound surface on the μIDEs before the sample and probe binding and enzymatic metallization reaction steps occur. The rest of the assay is performed as previously described, except that the probe solution contains only HRP-labeled anti-human IgG probe. Interestingly, this second assay scheme results in more than 100× further enhancement of sensitivity of detection. In addition, it eliminates the need for addition of AuNP-labeled probes and thus simplifies the assay. This assay can thus quantify anti-S IgG from serum sample volumes lower than 1 μL, and with the second scheme, at a dilution up to ˜10,000-fold.

Longer metallization times can result in increased sensitivity or lower limit of detection up to a point. The optimum metallization time is ˜8 min. As described above, this makes use of antigens immobilized on a nanostructured, catalytic AuNP-bound surface and its synergistic effect with enzyme-labeled probes to generate an enhanced, high density silver metallization to transduce specific probe-binding events as a change in electrical resistance.

The chip disclosed herein can be used as a miniaturized platform for high throughput screening of clinical samples from serum samples as low as 0.1 μL. Regarding the film, two layers of PDMS are laser-cut and reversibly sealed on the chip. The first layer includes four microwells which are aligned on the μIDEs and used for immobilization of different antigens. The second layer includes a larger well covering all the smaller wells for sharing sample and all further reagents between the smaller wells.

To investigate the possibility of multiplexed detection from a single sample drop, the model biotin-SA chemistry is used again. Biotin-BSA along with AuNPs is immobilized as the positive control on a different pair of μIDEs on each of four multiplexed microchips while the remaining three pairs of μIDEs on each microchip are coated with BSA and AuNPs as the negative controls. HRP-SA probe and silver substrate solutions are then sequentially added to the bigger sample microwells on each microchip so as to cover all four μIDEs. After metallization, washing and drying, deposition is observed only on positive control microwells, and no metallization is on negative control microwells. All positive controls showed low measured resistance and negative controls remained as open circuits. This shows that although the sample, probe and silver substrates are all shared as a single drop between smaller microwells, metallization occurs locally and remain on the surface of microwells which are coated with biotin-BSA and did not diffuse away or attach to other microwells. In this transduction technique, the enzymatic metallization is localized to where the enzyme-labeled probe is bound and further is dry-stable after washing as well. This result establishes a unique ability of this transduction for multiplexed enzyme amplified electronic detection of biomarkers from a single small drop of sample without any concern for crosstalk of signals from adjacent microwells.

The layout and reconfigurability of the multiplexed microchip and resealable thin-film PDMS microwell layer design allow different formats of multiplexing using the same microchip design. For instance, one isotype of antibodies (e.g. IgG) against different viral antigens can be detected by immobilizing different antigens on the four different μIDEs and using a probe against the specific isotype of antibody. Alternatively, different isotypes of antibodies (e.g. IgG and IgM) against multiple antigens can also be detected using different isotype-specific probes.

Thus, this multiplexed microchip assay successfully measures, from a single drop of serum sample, an antigen-specific antibody fingerprint that can differentiate between healthy, COVID+, and vaccinated samples using two different antigens and a probe against human IgG. Multiplexed detection of different antibody isotypes (e.g. IgG and IgM) can provide a more comprehensive insight on the status of infection. IgM antibodies are known to be the first antibodies produced by immune response and began to decline at week 3 of the illness while IgG antibodies are produced later and are detectable for longer periods.

A smartphone application, based on the Android platform, can be provided which communicates with the impedance analyzer via Bluetooth and acquires, plots, stores and communicates the data to cloud storage platforms. This enables the user to perform measurements through the app while the multiplexed chip is connected to the impedance analyzer. The app also shows the measured impedance values as numerical values in a bar graph or as an impedance heat map overlaid on the layout of the multiplexed chip.

In addition, the frequency of impedance measurement can also be tuned or impedance spectra over multiple frequencies can be measured in order to optimize the signal to background ratio. In addition, the multiplexable nature of this technique, demonstrated here using a 4-plex assay, also provides the opportunity for development of fully integrated POC automated electronic microarray systems for massively multiplexed detection of larger numbers of biomarkers. Multiplexed and quantitative biomarker measurements can enable more accurate diagnostic and prognostic monitoring in several contexts. Given the general applicability of the transduction technique to any binding-based assay, detection of COVID-19 neutralizing antibodies to monitor vaccine efficacy and detection of other biomarkers such as antigens, DNA, and even whole pathogens or cells for diagnosis and monitoring of infectious or other diseases are among a few of the many useful future applications possible.

For the microfabrication of μIDEs, glass wafers (100 mm diameter, 0.5 mm thickness) are cleaned in acetone and isopropyl alcohol for 5 minutes each, then cleaned in piranha solution for 20 minutes. Photoresist is spin coated and baked at 150° C. for 2.5 minutes. Standard photolithography process including UV exposure and development is done. After 30 seconds of plasma de-scum, 10 nm of titanium followed by 100 nm of gold is deposited via e-beam evaporation. Liftoff is performed by sonication in acetone for 5 minutes, and then the chips are rinsed with isopropyl alcohol and diced using a dicing saw.

For the PLL coating process, microfabricated chips are cleaned with 10% NaOH/60% reagent alcohol in deionized (DI) water for 2 hours followed by rinsing with DI water thoroughly. Next, chips are dipped in 30% PLL in 30 mM PBS for 30 minutes. Then, chips are rinsed with DI water and dried with centrifuging for one minute. PLL-coated chips are stored under vacuum in a desiccator at room temperature.

For PDMS preparation, PDMS film with a thickness of 0.1 mm is laser cut with a pattern of wells for each specific design of microfabricated chip and then soaked in a solution of 5% alconox in DI water for 30 minutes followed by rinsing with DI water. After air drying the PDMS films, tape is used to remove any remaining dust or particles before visually aligning and reversibly sealing with chips.

For multiplexing, any number of sets comprising a first and second electrode pair can be disposed on the same plate. Each pair being electronically isolated, each pair can be used to detect a distinct biomarker.

For the Biotin-BSA HRP-SA Model Assay, 1 mg/mL biotin-BSA in PBS is added to each well and incubated for one hour. All incubations are performed in a humidified chamber at room temperature unless otherwise specified. Next, the chip is blocked with 10% BSA in 0.1% Tween20 in PBS (0.1% PBST) for 30 minutes and washed with 0.1% PBST and PBS. All washing steps are performed by placing the chip in a 10 cm Petri dish filled with washing buffer on a plate shaker at 55 RPM for 10 minutes. Then, the chip is dipped in DI water and centrifuge-dried for one minute. Probe solutions, consisting of [1:400] HRP-SA, or [1:3] GNP-SA, or [1:400] HRP-SA and [1:3] GNP-SA are prepared in 1.5 mg/mL BSA in 0.05% PBST. Next, probe added to each well and incubated for 1 hour. After two washes with 0.1% PBST and one wash with PBS, the chip is dipped in DI water and centrifuge-dried for one minute.

For Serum Sample Dilution and Addition for Immunoassays, the custom laser-cut PDMS microwells used here hold between 1.5-3 μL each to ensure full surface coverage of the well and to avoid overflow. For all serum-based assay results reported here, a minimum sample dilution of 1:100 is used here, except for the serial dilution curve starts at a 1:10 serum dilution. To create the 1:100 diluted sample, 0.1 μL of each serum sample is initially diluted with 9.9 μL of assay buffer (of 0.01% BSA in 0.05% PBST) in a separate tube. Then 3 μL each of this diluted serum sample is directly added into the microwells.

For Silver metallization and impedance measurements, equal volumes of each substrate component of three-part EnzMet substrate solutions are sequentially added and incubated for 4 minutes, 4 minutes and 8 minutes respectively. Upon completion of the third development time, chips are dipped in DI water to stop the silver metallization reaction and spin dry to record dry phase impedance measurement of deposited silver metal. Impedance measurements are performed by measuring impedance across each pair of IDEs using the custom-built portable impedance analyzer which uses an applied voltage of 100 mV at a frequency of 10000 Hz.

For Integration of AuNPs in immunoassay, the AuNPs are integrated in the assay of COVID-19 either by mixing HRP-labeled probes with AuNP-labeled probes or via direct immobilization of AuNPs along with the antigen on the surface of the plate. This achieves the goal of creating a catalytic or nucleating surface. For the first scheme, a solution of 50 tg/mL of each COVID-19 antigen (S, N) or control proteins (BSA, AG) are prepared in PBS and added to each well and incubated overnight at 4 C. Next, the chip is blocked, washed and dried as above. 0.1 μL of each serum sample is diluted by 9.9 μL of 0.01% BSA in 0.05% PBST in a separate PCR tube. Then, 3 μL of each diluted serum sample is added to their corresponding wells. Then, samples are added to the wells and incubated for one hour, followed by washing and drying again. A probe solution consisting of [1:400] goat HRP-anti human IgG and [1:6] goat AuNP-anti human IgG in 1.5 mg/mL BSA in 0.05% PBST is prepared, and this mixture is added to each well. After one hour incubation, the chip is washed, dried and the silver metallization step is completed as above.

For the second scheme, [1:60] AuNPs in 0.5 mM PBS is added to each well and incubated in for one hour. Next, the chip is washed twice with PBS, dipped in DI water and centrifuge dried. Antigen immobilization and sample incubation is performed as above. A probe solution consisting of [1:800] mouse HRP-anti human IgG in 1.5 mg/mL BSA in 0.05% PBST is prepared and added to each well. After one hour incubation, the chip is washed and dried.

Then, the silver metallization reaction is completed, and the measurement of the electrical property is performed as disclosed herein.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

1. A composition comprising:
a molecule of interest configured to bind with a target molecule in a biological sample;
a probe molecule configured to bind with the target molecule;
a first metal particle configured to bind with the probe molecule; and
a second metal particle configured to amplify a binding of the first metal particle.

2. The composition of claim 1 further comprising a buffer;
wherein the first metal particle comprises at least one of a silver nanoparticle, a gold nanoparticle, an iron nanoparticle, silver, gold, iron, or platinum.

3. The composition of claim 2, wherein the biological sample comprises at least one of serum, whole blood, saliva, urine, or cerebrospinal fluid; and
wherein the second metal particle comprises at least one of gold, platinum, or iron oxide.

4. The composition of claim 3, wherein at least one of:
the molecule of interest comprises at least one of antigen, an epitope, an antibody, protein, DNA, RNA, a virus, a bacterium or a mammalian cell;
the target molecule comprises at least one of an antibody, a paratope, a protein, DNA, RNA, a virus, a bacterium, or mammalian cell; and
the probe molecule comprises at least one of horseradish peroxidase or alkaline phosphatase.

5.-8. (canceled)

9. The composition of claim 4, wherein the buffer is a phosphate buffer having a pH of from about 6.0 to about 7.0.

10. (canceled)

11. A method comprising:
forming an electrical connection comprising the composition of claim 1 between a first electrode and a second electrode; and
measuring an electrical property of the electrical connection;
wherein the electrical property is indicative of the presence or absence of a biomarker.

12. The method of claim 11, further comprising:
forming the electrical connection by:
disposing the molecule of interest disposed between the first electrode and the second electrode;
binding the molecule of interest to the target molecule;
incubating the target molecule in a probe solution comprising the probe molecule to bind the probe molecule to the target molecule;
binding the first metal particle to the probe molecule;
amplifying the forming of the electrical connection with the second metal particle; and
modifying the electrical property of the electrical connection.

13. The method of claim 12, wherein:
binding the molecule of interest to the target molecule comprises incubating the molecule of interest in a sample solution comprising the biological sample; and
binding the first metal particle to the probe molecule that is bound to the target molecule comprises enzymatically depositing the first metal particle on the probe molecule.

14. The method of claim 13, wherein at least one of:
incubating the molecule of interest in the sample solution lasts for approximately 1800 seconds at approximately room temperature; or
incubating the target molecule in the probe solution lasts for approximately 3600 seconds at approximately room temperature.

15.-18. (canceled)

19. The method of claim 12 further comprising:
disposing the first electrode and second electrode on a plate;
wherein:
the probe solution further comprises a second metal particle; or
the plate has on its surface the second metal particle.

20. The method of claim 19, wherein the second metal particle is gold.

21.-22. (canceled)

23. The method of claim 11, wherein measuring the electrical property of the electrical connection comprises:
measuring an impedance across the electrical connection with a lock-in amplifier.

24. (canceled)

25. The method of claim 23, wherein measuring the impedance comprises sampling at approximately 100,000 samples per second at one or more frequencies.

26. The method of claim 25, wherein the one or more frequencies are between approximately 45 kilohertz and 35 megahertz.

27. A system comprising:
the composition of claim 1;
a first electrode;
a second electrode; and
an electrical property sensor in electrical communication with the first electrode and the second electrode;
wherein the electrical property sensor is configured to measure an electrical property of the composition.

28. (canceled)

29. The system of claim 27 further comprising:
a plate;
wherein the first electrode and the second electrode are disposed on the plate within a well configured to contain liquid.

30. The system of claim 29, wherein at least one of:
the well is formed in a film;
the plate is polylysine coated glass; or
the plate has on its surface the second metal particle.

31.-34. (canceled)

35. The system of claim 30 further comprising:
a processor configured to determine a concentration of the target molecule in the biological sample based on an impedance across the electrical connection.

36. The system of claim 27, wherein at least one of:
the electrical property sensor comprises a lock-in amplifier;
the molecule of interest comprises at least one of an antigen, an epitope, an antibody, protein, DNA, RNA, virus, bacteria, or mammalian cell;
the target molecule comprises at least one of an antibody, a paratope, a protein, DNA, RNA, a virus, a bacterium, or a mammalian cell;
the biological sample comprises at least one of serum, whole blood, saliva, urine, or cerebrospinal fluid;
the first metal particle comprises at least one of a silver nanoparticle, a gold nanoparticle, an iron nanoparticle, silver, gold, iron, or platinum;
the second metal particle comprises at least one of gold, platinum, or iron oxide; or
the probe molecule comprises at least one of horseradish peroxidase or alkaline phosphatase.

37.-46. (canceled)

47. The system of claim 27, wherein the first electrode is an interdigitated electrode comprising a first plurality of fingers;
wherein the second electrode is an interdigitated electrode comprising a second plurality of fingers;
wherein at least one of the first plurality of fingers is adjacent to at least one of the second plurality of fingers and separated by a gap; and
wherein the gap is approximately 5 micrometers.

48. (canceled)
